(19) United States
(12) Patent Application Publication
(10) Pub. No.: US 2024/0296957 A1
(43) Pub. Date: Sep. 5, 2024

(54) METHOD AND SYSTEM FOR THE GENERATION AND ANALYSIS OF BIOMECHANICAL DATA

(71) Applicant: B-TEMIA INC., St-Augustin-de-Desmaures, (CA)

(72) Inventors: Marcus BROOKSHAW, St-Augustin-de-Desmaures (CA); Nathaniel ZOSO, St-Augustin-de-Desmaures (CA); Stéphane BÉDARD, St-Augustin-de-Desmaures (CA)

(21) Appl. No.: 18/268,823

(22) PCT Filed: Dec. 21, 2021
(86) PCT No.: PCT/CA2021/051866

§ 371 Date: Jun. 21, 2023

Related U.S. Application Data
(60) Provisional application No. 63/129,543, filed on Dec. 22, 2020.

(51) Int. Cl. G16H 50/30; A61B 5/00; A61B 5/11; G16H 50/70

(52) U.S. Cl. CPC G16H 50/30; A61B 5/112; A61B 5/1122; A61B 5/6812; A61B 5/7203; A61B 5/7275; G16H 50/70

(57) ABSTRACT

A system and method for generating a physiological assessment of a user from biomechanical data gathered from the user. The method comprises the steps of acquiring biomechanical data of the user. generating trajectory data of the user using the acquired biomechanical data. classifying the trajectory data into variables of interest data according to a set of key biomechanical features, rejecting variables of interest data that do not fit a pre-determined acceptance criteria. extracting key features of the non-rejected variables of interest data: calculating a current cumulative risk data based on 1) the extracted key features: 2) a user profile covariate data: and 3) the variables of interest data, and generating the physiological assessment of the user using the current cumulative risk data.

### CROSS-REFERENCE TO RELATED APPLICATIONS

This application claims the benefits of U.S. provisional patent application No. 63/129,543 filed on Dec. 22, 2020, which is herein incorporated by reference.

### TECHNICAL FIELD

The present disclosure relates to a method and system for the generation and analysis of biomechanical data.

### SUMMARY

The present disclosure provides a method for generating a physiological assessment of a user from biomechanical data gathered from the user, the method comprises the steps of:
acquiring biomechanical data of the user;
generating trajectory data of the user using the acquired biomechanical data;
classifying the trajectory data into variables of interest data according to a set of key biomechanical features;
rejecting variables of interest data that do not fit a pre-determined acceptance criteria;
extracting key features of the non-rejected variables of interest data;
calculating a current cumulative risk data based on the extracted key features, a user profile covariate data and the variables of interest data; and
generating the physiological assessment of the user using the current cumulative risk data.

The disclosed method may further comprise the step of:
sorting and labeling the trajectory data into discrete segments; and
filtering the discrete segments of trajectory data so as to reduce a number of individual frames and remove noise;
wherein the step of classifying the trajectory data into variables of interest data according to a set of key biomechanical features is performed on the filtered discrete segments of trajectory data.

The disclosed method may further comprise the step of formatting and simplifying the risk data of the assessment of the user for presentation to the user and/or adding biomechanically derived information to the assessment of the user.

The biomechanical data of the user can be acquired from biomechanical sensors positioned on the user and may be in the form of inertial and angular sensors positioned on a lower-body orthotic device worn by the user. The biomechanical sensors may be positioned, for example, at locations corresponding to the hip joints, knee joints, pelvic region, thighs, and feet of the user.

The present disclosure further provides a system for generating a physiological assessment of a user from biomechanical data gathered from the user, which comprises:
biomechanical sensors configured to observe associated user body segment kinematics;
a processor in communication with the plurality of biomechanical sensors, the processor having an associated memory comprising instructions stored therein that when executed on the processor perform the steps of:
receiving biomechanical data of the user from the plurality of biomechanical sensors;
generating trajectory data of the user using the acquired biomechanical data;
classifying the trajectory data into variables of interest data according to a set of key biomechanical features;
rejecting variables of interest data that do not fit a pre-determined acceptance criteria;
extracting key features of the non-rejected variables of interest data;
calculating a current cumulative risk data based on the extracted key features, a user profile covariate data and the variables of interest data; and
generating the physiological assessment of the user using the current cumulative risk data.

The memory may comprise further instructions stored therein that when executed on the processor further perform the steps of:
sorting and labeling the trajectory data into discrete segments; and
filtering the discrete segments of trajectory data so as to reduce a number of individual frames and remove noise;
wherein the step of classifying the trajectory data into variables of interest data according to a set of key biomechanical features is performed on the filtered discrete segments of trajectory data.

The memory may also comprise further instructions stored therein that when executed on the processor further perform the steps formatting and simplifying the risk data of the assessment of the user for presentation to the user and/or adding biomechanically derived information to the assessment of the user.

The disclosed system may further comprise:
a reference database containing key biomechanical measurements and associated physiological determinant factors, from peer-reviewed academic publications; and
an outcome database containing linked key biomechanical measurements and associated statistically established outcomes, from user monitoring and experimentation.
wherein the key biomechanical features are obtained by forming an indexed combination of curated data from the reference database and the outcome database:

The biomechanical sensors may include inertial and angular sensors positioned on a lower-body orthotic device worn by the user, and the system may also include a lower-body orthotic device configured to be worn by the user, wherein the biomechanical sensors include inertial and angular sensors positioned on the lower-body orthotic device. The biomechanical sensors may be positioned, for example, at locations corresponding to the hip joints, knee joints, pelvic region, thighs, and feet of the user.

### BACKGROUND

Biomechanical observations can reveal a wide variety of pathologies, however, access to this type of diagnostic testing is limited by the need for specialized laboratory equipment and inability to make discrete observations over time during daily activities. Existing technology (step counters, fitness apps on smart devices, etc.) allow for discrete observations over time, however they do not have sufficient sensors to track multiple body segments, limiting their use to gross measures (step count) that fail to capture the user's biomechanics, and therefore cannot give an accurate physiological assessment of the user's wellbeing Tracking biomechanical symptoms of movement disorders (e.g., Parkinson's Disease, Multiple Sclerosis, Ataxia, etc.) and other illnesses with gait symptoms (e.g., advanced knee or hip osteoarthritis, myopathologies, age-related strength deficits, etc.) can reveal key details of general health status and disease progression, and even predict future fall risk.

As a result, there is a need for a method and system for the generation and analysis of biomechanical data with the ability to accurately capture key biomechanical details (e.g., positions and timing of footsteps, joint motions, and posture) that can be used continuously in the home and community environment to physiologically assess the user's state.

### BRIEF DESCRIPTION OF THE FIGURES

Embodiments of the disclosure will be described by way of examples only with reference to the accompanying drawings, in which:

FIG. 1 is a schematic representation of the system for the generation and analysis of biomechanical data in accordance with the illustrative embodiment of the present disclosure;

FIG. 2 is a flow diagram depicting the process for determining the linked biomechanical and physiological determinants in accordance with the illustrative embodiment of the present disclosure;

FIG. 3 is a flow diagram of the process for the generation and analysis of biomechanical data for informing a physiological determinant-based analysis of an individual in accordance with the illustrative embodiment of the present disclosure; and

FIG. 4 is a flow diagram of the process for the creation of a physiological assessment of an individual based on their biomechanical data in accordance with the illustrative embodiment of the present disclosure.

Similar references used in different Figures denote similar components.

### DETAILED DESCRIPTION

Generally stated, the non-limitative illustrative embodiment of the present disclosure provides a method and system whose function is to generate a physiological measurement from the generation and analysis of biomechanical data.

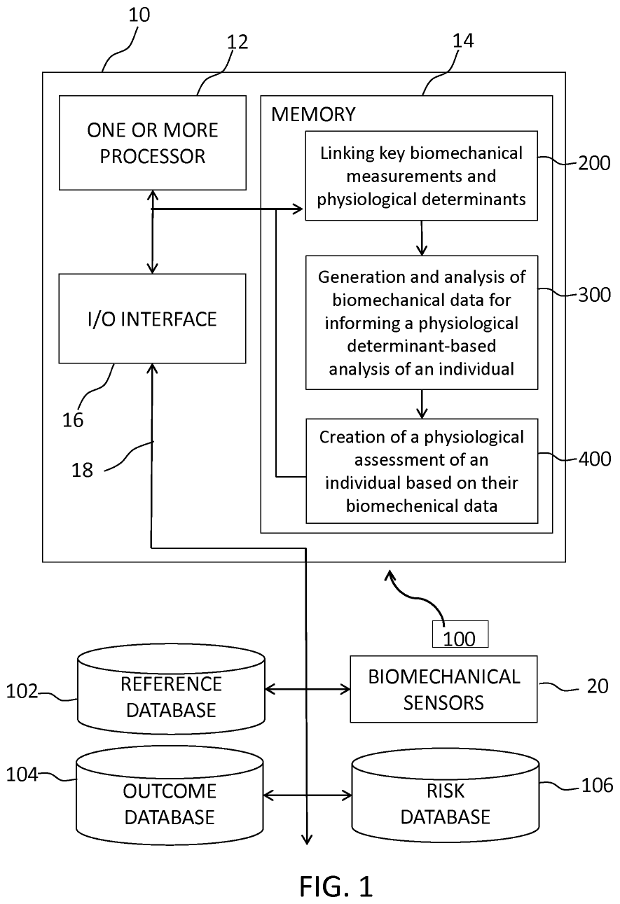

Referring to FIG. 1, the system for the generation and analysis of biomechanical data 100 includes one or more processor 12 with an associated memory 14 comprising instructions stored thereon, that when executed on the one or more processor 12, perform the steps of processes 200, 300 and 400, which processes will be further described below, and an input/output (I/O) interface 16 for communication with a plurality of biomechanical sensors 20, as well as a reference 102, an outcome 104 and a risk 106 databases, through a communication link 18, which may be wired, wireless or a combination of both. The biomechanical sensors 20, for example inertial and angular sensors, are configured to observe associated user body segment kinematics in order to provide mechanical and biomechanical information.

The biomechanical sensors 20 may be provided on an orthotic device, an example of which is disclosed in International Patent Application PCT/CA2021/051846 entitled “LOAD DISTRIBUTION DEVICE FOR IMPROVING THE MOBILITY OF THE CENTER OF MASS OF A USER DURING COMPLEX MOTIONS” filed on 18 Dec. 2021. In the disclosed orthotic device, biomechanical sensors are positioned on a pelvic support belt, thigh support elements, hip joint actuators, knee joint actuators and feet of the user.

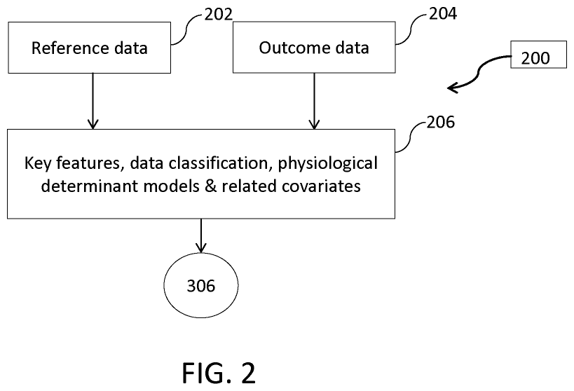

Referring now to FIG. 2, there is shown a flow diagram of the process for linking key biomechanical measurements and physiological determinants 200 executed by the one or more processor 12 (see FIG. 1) in accordance with an illustrative embodiment of the present disclosure. Steps of the process 200 are indicated by blocks 202 to 206.

The process 200 starts at block 202 where the reference database 102 of key biomechanical measurements (e.g., step length, hip trajectory, activity classification, etc.) and their associated physiological determinant factors (e.g., fatigue, falls, progression of gait-freezing-related disease symptoms, etc.) is accessed. The reference database 102 is maintained using peer-reviewed academic publications as a basis.

Similarly, at block 204, the outcome database 104 linking key biomechanical measurements and statistically established outcomes is accessed. The outcome database 104 is constructed through user monitoring and experimentation. For example, the outcome database 104 may link covariance in step-length between each leg of a user with disease progression among persons with dementia, variance in step-width with falls in elderly persons, etc.

Finally, at block 206, an ordered set containing key biomechanical features (e.g., spatiotemporal gait variables, postural sway, etc.), associated classification criteria (e.g., cut-off for asymmetry in step length being ≤20%, minimum 10 step consecutive gait cycles for a spatiotemporal calculation, etc.), models of physiological determinants of risk (e.g., age ≥65 and presence of knee extensor asymmetry ≥10% linked to higher fall probability, diagnosis of multiple sclerosis reduces likelihood that step-width variability is indicative of increased gait disfunction, etc.), and a list of known covariates (e.g., age, sex, disease diagnosis, relative frequency and types of personal activity, detected changes in activity levels over time, spasticity, strength, balance, and timed functional testing scores; presence of cognitive impairment; elapsed time since accident or disease diagnosis; height; weight; body mass index) is constructed as an indexed combination of curated data taken from the reference database 102 and the outcome database 104.

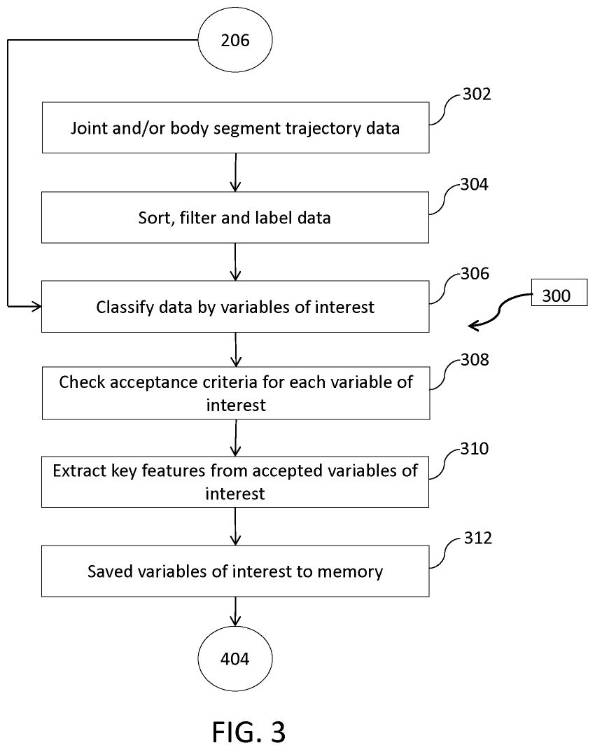

Referring to FIG. 3, there is shown a flow diagram of the process for the generation of biomechanical data for informing a physiological determinant-based analysis of an individual 300 executed by the one or more processor 12 (see FIG. 1) in accordance with the illustrative embodiment of the present disclosure. Steps of the process 300 are indicated by blocks 302 to 312.

The process 300 starts at block 302 where joint and/or body segment trajectory data of a user is determined, for example using a gait profiler such as disclosed in International Patent Application WO 2018/137016 A1 entitled “Gait Profiler System and Method” filed 25 Jan. 2017, and the acquired mechanical and biomechanical information from the biomechanical sensors 20.

At block 304, the trajectory data (e.g., 3D acceleration data of body segments and/or angular data from joints) is sorted and labeled into discrete segments, and then the results are filtered to reduce the number of individual frames and remove noise. The trajectory data can include joint angles; 1^{st}, 2^{nd}, or 3^{rd} order rate of change of joint angles; anterior-posterior, medio-lateral, or inferior-superior acceleration of the torso, pelvis, thigh, shank, or foot. For example, the 3^{rd} order rate of change of hip position (i.e., jerk) is a good indicator for detecting changes in postural sway for persons with medial (or lateral) ligament instability but there are situations where the change in knee angle can be used because the individual has knee stiffness, and the knee excursion is reduced by spasticity). In an alternative embodiment, ground reaction force data could also be used.

Then, at block 306, the filtered, sorted and labeled data are classified into variables of interest data according to the key biomechanical features of the ordered set from block 206 of FIG. 2. The variables of interest data are processed biomechanical data associated with specific times, postures, and/or activities (e.g., mean percentage of gait cycle spent in double stance during walking at preferred speed). These data are linked to key biomechanical features (e.g., double stance times) and may include, for example, knee, hip, or ankle joint excursion, posture and postural change over a period of time or during specific activities (e.g., walking, jogging, running, transfer activities, obstacle avoidance, weightbearing activities, athletic activities, and other activities involving the lower body); temporal and spatial characteristics of foot positioning (e.g., step time, swing time, stride time, stance time, single and double support time, step length, stride length, step width, cadence, gait speed, stride speed).

At block 308, the resultant variables of interest data are examined according to pre-determined acceptance criteria so as to reject data that do not fit the expected magnitude, shape, or trend over time based on physiological determinants associated with the specific variable of interest. The pre-determined acceptance criteria are cut-off requirements for accepting specific variables of interest as meaningful (e.g., flag an increase in double support time if a statistically significant increase in double-support phase of walking month over month is detected for consecutive months, mean increase is more than 1% of gait cycle, and if user meets risk criteria for balance/movement disorders and/or is diagnosed with a balance/movement disorder).

At block 310, each segment of biomechanical data with accepted variables of interest are then processed and segmented to extract only the key features. The key features which are biomechanical features associated with the variables of interest (e.g., double stance time) may include, for example, mean asymmetry in step length during walking during morning, change in postural sway during sit-to-stand motion across the day, paired angular data as a measure of joint coordination (e.g., hip angle versus knee angle, left knee angle versus right knee angle), statistical treatment of variables of interest data (e.g., mean and variability of walking speed, average coefficient of correspondence between knee and hip joint across strides during walking; mean, standard deviation, variance, maximum and minimum values, and coefficient of variation in step width, stride length, step time, symmetry in step characteristics, joint excursion, and gait phases (swing, single support stance, double support stance) between right and left leg; mean and peak change in sagittal plane knee, hip, or ankle excursion across swing or stance phases of walking gait; mean change or coefficient of variation in posture during specific activities or parts of activities (e.g., angle of lower back during weight-acceptance portion of chair-rise, toe-in angle and knee vargus/valgus during stance phase of gait, squat depth, mean and peak center of mass excursion over base of support during chair rise), gait phase parameters (e.g., single-support time, double-support time, total stance time, swing time, stance/swing ratio, symmetry in the preceding parameters), estimation of joint power based on segment and/or joint angle in specific postures (e.g., acceleration of thigh or angular acceleration of knee during propulsion phase of sit-to-stand, angular acceleration of hip during toe-off phase of walking), estimations of joint stability (e.g., rate of change of knee flexion angle during weight-acceptance phase of walking, variability in ankle plantarflexion during loading-response phase of stance).

Finally, at block 312, the accepted variables of interest data are then archived and saved in memory 14.

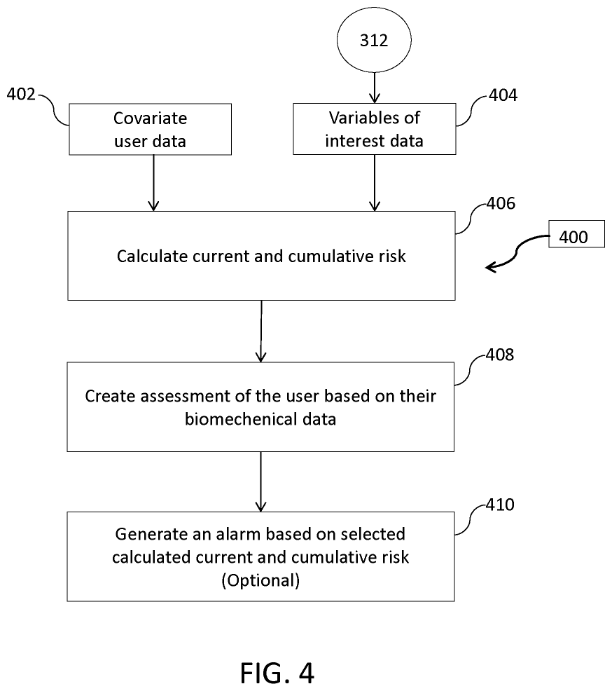

Referring to FIG. 4, there is shown a flow diagram of the process for the creation of a physiological assessment of an individual based on their biomechanical data 400 executed by the one or more processor 12 (see FIG. 1) in accordance with the illustrative embodiment of the present disclosure. Steps of the process 400 are indicated by blocks 402 to 410.

The process 400 starts at block 402 by accessing user profile covariate data, which is information that relates to the biomechanical features and risk data in a mitigative or enhancing way (e.g., if a persons has been diagnosed with multiple sclerosis, based on the literature, it is expected that increases in double stance time accompany decline in gait and balance, alternately, if an individual is young (<50) and does not have diagnosed movement or balance disorders, a change in double stance time would be less significant, in general) and may also include age, disease status, etc., and at block 404, the variables of interest data from block 312 of FIG. 3.

Then, at block 406, current and cumulative risk is calculated based on the extracted key features, with the covariate data from block 402 and the variables of interest data from block 404, which provide contextual basis (e.g., trend in knee excursion angle symmetry during walking over last year, mean walking speed this morning, etc.). The current and cumulative risk data is formed from the risk database 106 of previous risk assessments ('current risk data' during previous timeframes), typically reviewed over monthly and yearly timeframes (e.g., double support time during walking measured each month, trend in double support time month over month, year over year), and may include, for example, reduction in chair transfer fall risk across 24-hour period due to improved postural sway and symmetry, decrease in step symmetry over last year indicating declining knee strength and increased fall risk, change in fall risk across 24-hour period due to reduction in postural sway in walking and transfer activities and/or excursion of center of mass over base of support during chair-rise, and/or variance in step-width, step-length, or symmetry; fatigue estimation based on variance in spatiotemporal gait parameters between morning and afternoon periods, trends in: apparent fatigue, mobility (quantity and quality of movements), user activity levels (quantity and types of activities), apparent disease progression (e.g., joint angle excursion during specific activities as a measure of spasticity, variance in foot positioning or reduction in walking speed or reduction in specific mobility tasks (e.g., use of stairs, fast walking, tight turning) as measures of change in mobility status and competence); short and long-term trends in measures of gait and movement quality as a measure of changes in health status based on key indices like age, sex, height, weight, body mass index, disease status, and levels of activity relative to literature-based expectations of peer-group.

At block 408, the process 400 generates a physiological assessment of the user based on their biomechanical data. The user assessment is created using the current and cumulative risk data from block 406, formatting and simplifying the risk data for presentation to the user (e.g., displaying a single fall risk assessment, trimming the number of significant figures in displayed numerical data) and adding biomechanically derived information/graphics (e.g., step and activity counts, change in step count over time).

Optionally, at block 410, an alarm may be generated based on selected current and cumulative risks identified at block 406 (e.g., elevated risk of fall).

In a first sample embodiment, the covariance of a user's step length and step times would be variables of interest to the physiological determinant ‘Fall risk’. Walking periods shorter than two meters or 10 gait cycles would be disregarded. Based on the key biomechanical measurements reference database 102, key covariates for these variables of interest would be the current trend in variability, time of day, hours of activity (fatigue), disease status, and age. An assessment of the user, generated at block 408 of FIG. 4, would include an assessment of fall risk based on the covariance of step length and time, as well as measures associated with balance (e.g., postural sway and rate of change of hip acceleration in from left-to-right during forward walking).

In a second sample embodiment, the kinematic data of a user wearing a joint measurement device on a knee orthosis may provide the input data at block 302 of FIG. 3 (e.g., angle, angular velocity, and angular acceleration of the knee joint, step timing and counts over activity). Non-walking periods and walking periods shorter than 10 gait cycles would be disregarded. Change in knee excursion angle during walking (individual walking periods, mean morning value, mean night value, and daily average) would be the variable of interest to the physiological determinant of ‘change in knee spasticity’. Based on reference data of block 202 of FIG. 2, previous measurements of mean knee excursion angle during walking, volume of walking, disease status, age, and local weather conditions would be the key covariates for the variable of interest. An assessment of the user, generated at block 408 of FIG. 4, would include an assessment of spasticity change based on change in mean knee excursion angle during walking over a daily, weekly, and monthly period, as well as measures associated with activity level (total steps, change in steps over time) and apparent impact of spasticity change (relationship between change in knee excursion angle with activity level).

In a third sample embodiment, the kinematic data of a user wearing a joint measurement device on an elbow orthosis may provide the input data at block 302 of FIG. 3 (e.g., angle, angular velocity, and angular acceleration of the elbow joint, timing of maximum and minimum joint angles, and timing of rest periods). Data would be formatted into repetitions and within the repetitions, into the four components of joint exercise motions (eccentric task, isometric/rest, concentric task, isometric/rest task). Time under tension would be calculated using the total elapsed time of static or dynamic loading for the flexion and extension motions. Joint motions outside of the exercise period would be removed. User feedback (audio/haptic/visual) could be provided on rest periods, status of repetitions numbers, and tempo. The consistency of exercise tempo, rest periods, angular excursion of repetitions, number of repetitions, and time under tension would be the variables of interest to the physiological determinant of exercise quality. Based on reference data of block 202 of FIG. 2, caffeine use, fatigue (volume of joint motion during current measurement period as well as in the day overall), and level of adaptation to the current routine (approximated using historical data for the individual) are covariates for the variable of interest. An assessment of the user, generated at block 408 of FIG. 4, would include an assessment of exercise quality based on exercise tempo, muscle time under tension, repetitions, and rest periods, as well as an overall quality metric calculated from the individual quality metrics.

It is to be understood that the generation and analysis of biomechanical data process disclosed therein may also be used for upper limb movements, as well as determine other types of risk factors.

Although the present disclosure has been described by way of particular non-limiting illustrative embodiments and examples thereof, it should be noted that it will be apparent to persons skilled in the art that modifications may be applied to the present particular embodiment without departing from the scope of the present disclosure.

1. A method for generating a physiological assessment of a user from biomechanical data gathered from the user, the method comprising the steps of:
acquiring biomechanical data of the user;
generating trajectory data of the user using the acquired biomechanical data;
classifying the trajectory data into variables of interest data according to a set of key biomechanical features;
rejecting variables of interest data that do not fit a pre-determined acceptance criteria;
extracting key features of the non-rejected variables of interest data;
calculating a current cumulative risk data based on the extracted key features, a user profile covariate data and the variables of interest data; and
generating the physiological assessment of the user using the current cumulative risk data.

2. The method of claim 1, further comprising the step of:
sorting and labeling the trajectory data into discrete segments; and
filtering the discrete segments of trajectory data so as to reduce a number of individual frames and remove noise;
wherein the step of classifying the trajectory data into variables of interest data according to a set of key biomechanical features is performed on the filtered discrete segments of trajectory data.

3. The method of claim 1, further comprising the step of formatting and simplifying the risk data of the assessment of the user for presentation to the user.

4. The method of claim 3, further comprising the step of adding biomechanically derived information to the assessment of the user.

5. The method of claim 1, wherein the key biomechanical features are obtained by forming an indexed combination of curated data from a reference database and an outcome database:
the reference database containing key biomechanical measurements and associated physiological determinant factors, and being constructed using peer-reviewed academic publications; and
the outcome database containing linked key biomechanical measurements and associated statistically established outcomes, and being constructed using user monitoring and experimentation.

6. The method of claim 1, wherein the biomechanical data of the user is acquired from biomechanical sensors positioned on the user.

7. The method of claim 6, wherein the biomechanical sensors include inertial and angular sensors positioned on a lower-body orthotic device worn by the user.

8-9. (canceled)

10. A system for generating a physiological assessment of a user from biomechanical data gathered from the user, comprising:
biomechanical sensors configured to observe associated user body segment kinematics;
a processor in communication with the plurality of biomechanical sensors, the processor having an associated memory comprising instructions stored therein that when executed on the processor perform the steps of:
receiving biomechanical data of the user from the plurality of biomechanical sensors;
generating trajectory data of the user using the acquired biomechanical data;
classifying the trajectory data into variables of interest data according to a set of key biomechanical features;
rejecting variables of interest data that do not fit a pre-determined acceptance criteria;
extracting key features of the non-rejected variables of interest data;
calculating a current cumulative risk data based on the extracted key features, a user profile covariate data and the variables of interest data; and
generating the physiological assessment of the user using the current cumulative risk data.

11. The system of claim 10, wherein the memory comprises further instructions stored therein that when executed on the processor further perform the steps of:
sorting and labeling the trajectory data into discrete segments; and
filtering the discrete segments of trajectory data so as to reduce a number of individual frames and remove noise;
wherein the step of classifying the trajectory data into variables of interest data according to a set of key biomechanical features is performed on the filtered discrete segments of trajectory data.

12. The system of claims claim 10, wherein the memory comprises further instructions stored therein that when executed on the processor further perform the steps of formatting and simplifying the risk data of the assessment of the user for presentation to the user.

13. The system of claim 12, wherein the memory comprises further instructions stored therein that when executed on the processor further perform the steps of adding biomechanically derived information to the assessment of the user.

14. The system of claim 10, further comprising:
a reference database containing key biomechanical measurements and associated physiological determinant factors, from peer-reviewed academic publications; and
an outcome database containing linked key biomechanical measurements and associated statistically established outcomes, from user monitoring and experimentation.
wherein the key biomechanical features are obtained by forming an indexed combination of curated data from the reference database and the outcome database:

15. The system of claim 10, wherein the biomechanical sensors include inertial and angular sensors positioned on a lower-body orthotic device worn by the user.

16. The system of claim 10, further comprising a lower-body orthotic device configured to be worn by the user, wherein the biomechanical sensors include inertial and angular sensors positioned on the lower-body orthotic device.

17-18. (canceled)
